(19) United States
(12) Patent Application Publication
(10) Pub. No.: US 2025/0387830 A1
(43) Pub. Date: Dec. 25, 2025

(54) REACTIVE OXYGEN SPECIES RESPONSIVE CLEAVABLE HIERARCHIAL METALLIC SUPRANANOSTRUCTURES

(71) Applicant: Northwestern University, Evanston, IL (US)

(72) Inventors: Dong Hyun Kim, Evanston, IL (US); Hyunjun Choi, Evanston, IL (US); Bongseo Choi, Evanston, IL (US)

(21) Appl. No.: 18/880,219

(22) PCT Filed: Jul. 3, 2023
(86) PCT No.: PCT/US2023/069591

§ 371 Date: Dec. 30, 2024

Related U.S. Application Data
(60) Provisional application No. 63/367,547, filed on Jul. 1, 2022.

(51) Int. Cl. B22F 1/068; B22F 1/07; B22F 9/24

(52) U.S. Cl. CPC B22F 1/068; B22F 1/07; B22F 9/24; B22F 2301/255; B22F 2304/054; B22F 2304/056; B22F 2304/058

(57) ABSTRACT

Hierarchical metallic supra-nanostructure (HMSN) comprising branches with alternating nanocrystals and nano-linkers, where the nanocrystals comprise gold and the nano-linkers comprise a second metal having a lower reduction potential than the nanocrystals, are disclosed. The nano-linkers may selectively dissolve in the presence of reactive oxygen species (ROS). Also disclosed are methods of, making and using the same.

### CROSS-REFERENCE TO RELATED APPLICATIONS

This application claims benefit of priority to U.S. Patent Application Ser. No. 63/367,547, filed Jul. 1, 2022, the contents of which is incorporated by reference in its entirety.

### STATEMENT REGARDING FEDERALLY SPONSORED RESEARCH

This invention was made with government support under 5R01CA209886-05 and EB026207 awarded by the National Institutes of Health. The government has certain rights in the invention.

### BACKGROUND OF THE INVENTION

Various reactive oxygen species (ROS) responsive organic ligands and polymer materials employing ROS responsive moieties have been investigated and utilized for ROS responsive cancer therapeutic applications. Recently, multifunctional ROS responsive nanocarriers which can perform therapeutic/imaging in a single platform have been paid much attention as a promising form of ROS responsive nanocarriers. Hybrid organic/inorganic nanocarriers integrating ROS responsive moiety and multifunctional inorganic nanoparticles have been suggested for a multifunctional ROS responsive nanocarriers. However, combining multiple functions into one carrier system remains a challenge and limits their broad cancer therapy applications. In addition, non-degradable inorganic nanoparticles in the hybrid platforms may induce side effects with low level of clearance. An effective ROS responsive degradable nanoparticle may be beneficial for ROS triggered cancer therapeutics that can result in both enhanced treatment outcome and reduced side effects.

### BRIEF SUMMARY OF THE INVENTION

Disclosed herein are hierarchical metallic supra-nanostructures (HMSNs) and methods of making and using the same. A HMSN comprises branches with alternating nanocrystals and nano-linkers, wherein the nanocrystals comprise a first metal and the nano-linkers comprise a second metal having a lower reduction potential than the nanocrystals. The nano-linkers may selectively dissolve in the presence of reactive oxygen species (ROS). In some embodiments, first metal is gold and/or the second metal is silver. In some embodiments, the HMSN may be conjugated to an agent, such as a photosensitizer.

In another aspect of the technology, methods of preparing a HMSN are provided. The method may comprise preparing a reaction mixture by adding a nanocrystal precursor and nano-linker precursor to a metal cholate solution; adding a reducing agent to the reaction mixture; and preparing a nanoparticle suspension from the reduced reaction mixture.

In another aspect of the technology, methods of treating a subject in need of a ROS-mediated therapy are provided. The method may comprise administering any of the HMSN described herein and the ROS-mediated therapy. Suitably, the ROS-mediated therapy may comprise irradiating the subject. In some embodiments, the subject is in need of a treatment for cancer. Suitably, the methods described herein may induce induces apoptosis, senescence, or cell cycle arrest in cancer cells.

In another aspect of the technology, methods of degrading HMSN are provided. The method may comprise contacting the HMSN with a ROS. Upon contact, the ROS will selectively degrade the nano-linkers, resulting in the generation of a multiplicity of smaller nanoparticles. In some embodiments, the HMSN are contacted with ROS in vivo.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Panel a) shows representative transmission electron microscopic image of HMSN (the size bar represents 100 nm) and structural information of HMSN based on observation by TEM. Panel b) shows mean particle size and distribution of HMSN obtained from dynamic light scattering (DLS). Panel c) shows size and distribution measurement using DLS after the incubation of HMSN with various concentration of H₂O₂ (0, 0.1 mM, 1 mM, 0.1, and 1 M), panel d) shows representative TEM images of HMSN samples collected at the predetermined incubation time (1 day, 4 days, 1 week, 2 weeks, and 4 weeks) with H₂O₂ (the size bar represents 100 nm). Panel e) shows a table showing particle size changes on the HMSN by treatment of H₂O₂ after predetermined time. Panel f) shows TEM images of small nanoparticles generated from HMSN after the incubation with H₂O₂ (the size bar represents 100 nm) and size information of small nanoparticles derived from HMSN based on more than 200 nanoparticles counted from TEM images.

FIG. 2: Panel a) shows scanning transmission electron microscope (STEM) images of HMSN after the incubation with H₂O₂ (gold and silver in images were colored with red and green color, respectively and the size bar represents 100 nm). Panel b) shows a graph of the silver to gold ratio calculated from the ROI quantification of each element. Panel c) shows Quantification of silver element released from HMSN by H₂O₂ up to 72 hours. Panel d) shows A schematic image of the cleavage mechanism of HMSN in the presence of ROS.

FIG. 3: Panel a) shows bright field images merged with DAPI signals of cells using confocal microscopy (black dots indicate the gold nanoparticles accumulated in cells). Panel b) shows confocal reflective microscopic images for the detection of gold nanoparticle in living cell after uptake of gold nanoparticles (HMSN and SGNP). Panel c) shows confocal laser scanning microscopic images for ROS quantification in live cells after uptake of gold nanoparticles (HMSN and SGNP). Panel d) shows quantification of green fluorescence (DCF2-DA) for measuring ROS generation of RAW 264.7 cells after predetermined time up to 7 days. Panel e) shows cell TEM images of RAW 264.7 macrophage cells co-incubated with HMSN. The images at bottom line were obtained from cell samples on same TEM grid from cells.

FIG. 4: Panel a) shows TEM images of HMSN loaded with Ce6 after the various irradiation doses by varying the exposure time (20, 40, and 60 min with 652 nm laser (120 mW/cm²). Panel b) shows measurement of hydrodynamic size after the treatment of 652-nm laser using DLS. Panel c) shows a schematic image introducing experimental set-up for evaluating the diffusion of nanoparticles after ROS treatment. Panel d) shows Images taken by a digital camera before/after inducing ROS after applying threshold function for determining the diffused distance of nanoparticles in tissue mimicking agarose hydrogel. Panel e) shows a measurement of the distance that HMSN traveled from the seeded point after the induction of ROS or not. Panel f) shows observation of contrast effect of gold nanoparticles (HMSN and SGNP) by computed tomographic images of tumor bearing mice treated with gold nanoparticles (HMSN and SGNP) and ROS inducing laser subsequently. Panel g) shows a comparison of CT contrast effect (maximum value in ROI of each CT slices) of HMSN before and after treatment of ROS in tumor.

FIG. 5: Panel a) shows comparative study of biodistribution profiles of gold nanoparticles (HMSN and SGNP) after 3 days and 30 days post injection. Panel b) shows elemental (Au) quantification in blood of each group. Panel c) shows pathological evaluation of HMSN on excretory organs (kidney, liver, and spleen) by hematoxylin-eosin staining. Yellow dotted circle in the images of kidney samples indicates glomerulus (GM). the portal veins (PV), bile ducts (BD), hepatic arteries (HA) were annotated with yellow arrows on the liver samples. The area of white pulp (WP) and red pulp (RP) was highlighted on the spleen sections.

FIG. 6: Scanning transmission electron microscope (STEM) images showing gold and silver components of branches in HMSN (Red: gold and Green: silver).

FIG. 7: STEM image and elemental analysis of small nanoparticles generated from ROS treated HMSN.

FIG. 8: Dark field microscopic images demonstrating ROS effect on gold nanoparticles in cells by inhibition assay using N-acetylcysteine (NAC) after uptake of gold nanoparticles (HMSN and SGNP).

FIG. 9: Panel a) shows X-ray contrast effect of HMSN in various concentration dispersed in agarose phantom for computed tomography. Panel b) shows the relationship between Hounsfield Unit (HU) and HMSN concentration (mg/ml).

FIG. 10: Panel a) shows CT images of HMSN and SGNP before and after PDT-ROS treatment. Panel b) shows CT signal intensity changes of tumors in each group.

FIG. 11: TEM images of HMSN after X-ray irradiation with various doses (0 to 100 gy) and the measurement of hydrodynamic size after X-ray irradiation using DLS. Ionizing radiation, which is another potential exogenous ROS source for the cancer treatment, showed the same treatment responsive structural deformation. When the X-ray was irradiated to HMSN suspension up to 100 Gy, dose-dependent deformation and degradation of HMSN was well observed in TEM images and DLS measurement.

FIG. 12: Cell viability test of Clone-9 normal liver cell line for evaluation of biocompatibility of HMSN in vitro.

FIG. 13: Hematological values of mice treated with IV injection of HMSN or SGNP after 1-week post-treatment. Samples (2.5 mg/kg, Au) were intravenously injected into C57BL/6 mice. After 1 week, the blood samples were collected using intracardiac blood collection. All samples were collected with a heparin coated tube and centrifuged (1,000 g, 15 min, 4° C.). Plasma samples were frozen at −80° C. An automatic chemistry analyzer (Fuji Film DRI-CHEM NX500i) was used to quantify the level of AST, ALT, ALP, CREA, TBIL, CHOL, TG, GLU, HDL, GGT, LDH-P, ALB, CPK, Ca, and NA/K in plasma.

### DETAILED DESCRIPTION OF THE INVENTION

Disclosed herein is a hierarchical metallic supra-nanostructure (HMSN) and methods of making and using the same. The HMSN have a hierarchical structure with the primary nanocrystal component alternating with the secondary nano-linker component. The HMSN may be utilized in a number of different applications, such as therapeutic treatments, sensing, or imaging.

As demonstrated by the Examples. the HMSN are reactive oxygen species (ROS) responsive at biological conditions. When the HMSN are contacted with ROS, the nano-linker component is selectively degraded. Selective degradation of the nano-linker is demonstrated for endogenous ROS as well as therapeutic exogenous ROS prepared by photodynamic therapy (PDT) and X-ray irradiation. ROS-degradation of the HMSN indicates faster clearance than spherical gold nanoparticles of comparable size.

The alternating nanocrystals and nano-linkers are arranged in branches emanating from a generally central region of the HMSN in multiple, straight primary branches. From the primary branches, there are multiple secondary branches. Demonstrated in the Example the primary branches may be about 4.0-7.0 nm and the thickness of the secondary branches may be about 3.0-6.0 nm. Other thicknesses of primary and secondary branches are also possible by controlling the conditions for HMSN formation.

At least one of either the nanocrystals or the nano-linkers components include a metal. In one embodiment demonstrated in the Examples, both the nanocrystals and nano-linkers are metallic.

Nanocrystals are defined as materials with a crystalline structure and having at least one dimension below 100, 50, 25, 20, 15, or 10 nanometers. The nanocrystals disclosed in the Examples include gold, silver, iron, platinum, palladium, and any divalent metal components. As demonstrated in the Examples, the HMSN displayed nanocrystals have an average particle size about 3.8 nm (ranged from 1 nm to 10 nm) following ROS degradation.

Nano-linkers are defined as materials with at least one dimension below 100, 50, 25, 20, 15, or 10 nanometers that is positioned between and links together two nanocrystals.

One aspect of the technology is the selective dissolution of the nano-linkers in the presence of reactive oxygen species (ROS). Reactive oxygen species (ROS) are defined as highly reactive, oxygen-containing molecules. ROS include peroxides, including hydrogen peroxide, superoxide, hydroxyl radical, singlet oxygen, and alpha-oxygen. The material for the nano-linker component is selected to have a lower reduction potential relative to the nanocrystal component, thereby allowing for selective ROS degradation. In the demonstrated Examples, the reduction potential of the nano-linkers is E=0.80 V and the reduction potential of the nanocrystal is E=1.50 V. In other embodiments, the relative difference of reduction potentials can be larger or smaller than the embodiment disclosed in the Examples. The nano-linkers demonstrated in the Examples include silver, platinum, palladium, and any divalent metal components having lower reducing potential values than the nanocrystals.

The relative proportion of nanocrystals to nano-linkers may be varied depending on the method of HMSN preparation and optional subsequent processing. For example, the ratio of nanocrystals to nano-linker may vary from about 12.0:1 to 0.5:1. As demonstrated in the Examples, the HMSN has a ratio of nanocrystals to nano-linker of 10.6:1 as determined from ICP-MS analysis.

Particle size is defined as a measurement at least one particle dimension, routinely diameter. As demonstrated in the Examples, the particle size of the HSMN may be measured by dynamic light scattering (DLS) and may be reported as a hydrodynamic size. Persons skilled in the art are aware that hydrodynamic size includes the electric dipole layer adhered to the surface of the particle as it moves through a liquid medium. In one embodiment the hydrodynamic size of the HSMN is about 200 nm. Other methods of measuring particle size utilized in the Examples include visualization by electron microscopy techniques, such as scanning transmission electron microscopy (STEM) and scanning electron microscopy (SEM). These methods do not measure hydrodynamic size.

In some embodiments the HSMN has a particle size of 20 nm to 1000 nm. In some embodiments the HSMN has an average particle size from no smaller than 20 nm to no larger than 800 nm.

In one embodiment, the HMSN may be a 2-dimensional structure. A 2-dimensional structure is defined as a substantially planar structure. In the context of HMSN, the 2-dimension structure may be indicated all branches of the HMSN lying in substantially the same plane. As observed in the microscopy studies of the HMSN demonstrated in the Examples, the branches of the HMSN extend from a central region of the structure and along the same plane. 2-dimensionality of the HMSN may be determined by transmission electron microscopy (TEM) or other suitable imaging techniques.

In some embodiments, the HMSN further comprises a conjugated agent. Exemplary agents include, without limitation, therapeutic agents, imaging agents, or sensing agents. In some embodiments, the therapeutic agent is intended for use with PDT, such as photosensitizers, or x-ray irradiation. The Examples demonstrate the conjugation of Ce6 to the HMSN nanostructure, but the agent conjugated to the HMSN are not particularly limited. Exemplary photosensitizers may also include, without limitation, porphyrins, phthalocyanines, indocyanine dyes, BODIPYs, diketopyrrolopyrrole (DPP), curcumin, Ru(II) complexes, Ir(III) complexes, Au(III) complexes, polyfluorene, polythiophene, black phosphorous, metal sulfide based photosensitizers, and on the like.

Various conjugation methods that are known in the art. may be utilized. In one embodiment, an agent is conjugation with EDC/NHS chemistry.

The synthesis of HMSN includes preparing a reaction mixture by adding a nanocrystal precursor and a nano-linker precursor to a metal cholate solution, adding a reducing agent to the reaction mixture, and preparing a nanoparticle suspension from the reduced reaction mixture.

The nanocrystal precursor is a compound that provides a source of the nanocrystal component. The nanocrystal precursor may be a compound comprising a metal, a metal ion, or metal salt. Exemplary nanocrystal precursors include HAuCl₄.3H₂O, AuCl, H₂PtCl₆ or Na₂PdCl₄.

The nano-linker precursor provides a source of the nano-linker component. The nano-linker precursor may be a compound comprising a metal, a metal ion, or metal salt. Exemplary nano-linker precursors include AgNO₃, H₂PtCl₆ and Na₂PdCl₄.

A metal cholate complex is defined as a cholate complexed with at least one metal. In some embodiments, the metal cholate may include or be prepared from sodium cholate, sodium deoxycholate, cholate, chenodeoxycholic acid, ursodeoxycholic acid, lithocholic acid, and bile acid. The role of the metal cholate nanocomplex concentrate both nanocrystal precursor ions and nano-linker precursor nanoparticles inside of cholic acid micelles to create ideal conditions for preferential Au crystal growth and the formation of multi-branched HMSN. In one embodiment, the nanocrystal precursor ions are Au ions and the nano-linker precursor nanoparticles are AgCl.

A reducing agent is defined as a chemical species that donates an electron to a recipient. In some embodiments, the reducing agent is 1-ascorbic acid, 4-aminophenol, tricthylamine (TEA). Glycine, indole, 1,4-phenylenediamin, tryptophan, sodium citrate, or pyridine. The role of the reducing agent is to trigger the formation of hierarchical supra-nanostructures.

A nanoparticle suspension is defined as a heterogenous mixture of solid nanoscale particles in a fluid wherein the particles do not settle out. In one embodiment, the nanoparticles can be aided to form a suspension by agitation. In another embodiment, surface functionalization of the nanoparticle to make the surface of particle more water stable maybe used to generate a suspension. In the Examples, both agitation at 350 rpm during nanostructure synthesis and surface functionalization with thiol-polyethylene glycol (PEG)-carboxylate are demonstrated.

While ROS are byproducts of normal cellular metabolism of oxygen, cancer tumor growth and malignant progression at high metabolic rate are induced with moderate ROS levels an increased antioxidant ability. High concentrations of ROS have been implicated in destroying, slowing, or halting cancer cell growth via cancer cell cycle arrest, senescence and apoptosis. For that reason, ROS-mediated cancer cell therapies are of interest for suppressing tumor burdens. ROS-mediated therapies include photodynamic therapy (PDT) or radiation therapies.

One application of the present disclosure utilizes ROS-mediated therapies to induced exogenous ROS to initiate the structural degradation of HMSN. The ROS-degraded HMSN results in a multiplicity of smaller nanoparticles that may result in faster clearance in vivo. In some embodiment, the multiplicity of nanoparticles has an average size of no smaller than 1 nm and no larger than 10 nm. In one embodiment demonstrated in Examples, the nanoparticles are about 3 nanometers as measured by STEM.

One further aspect of the disclosed technology is the treatment of a subject in need. As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with an ROS-mediated therapy. For example, a “subject in need of treatment” may include a subject having a cell proliferative disease, disorder, or condition such as cancer. In some embodiments, the cancer that is associated with deregulated redox homeostasis. In some embodiments the cancer may be skin cancer, oral cancer, esophageal cancer, liver cancer, colorectal cancer, breast cancer, renal carcinoma, lung cancer, brain cancer, bladder cancer, bile duct cancer, pancreatic cancer, or head and neck cancer.

The HMSN utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. In some embodiments, the pharmaceutical composition comprises the HMSN and one or more additional therapeutic agents.

The HMSN utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the HMSN may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Administration of the HMSN to the subject may be performed in combination of with a ROS-mediated therapy. ROS-mediated therapies are known in the art and may include, for example, PDT or x-ray therapy. In some embodiments, administering the ROS-mediated therapy comprises irradiating the subject. Irradiation of a subject in need may include irradiation with visible, infrared, near-infrared, and x-ray wavelengths with dosages selected for the desired ROS-mediated therapy. For example, where the ROS-mediate therapy includes the generation of ROS by irradiating a photosensitizer administered to the subject, the wavelength and dosage may be selected based on the photosensitizer and desired therapeutic outcome. Selections such as those may be determined by clinicians having appropriate expertise. The Examples demonstrate radiation may have a wavelength of about 652 nm and dosage ranging from 150 J/cm² to 450 J/cm² and X-ray radiation may be used up to 100 Gy, but the methods described herein are not limited to those in the Examples. In some embodiments, the radiation is near infrared (NIR) having a wavelength of 800-1500 nm

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Synthesis of HMSN and ROS Responsive Structural Degradation

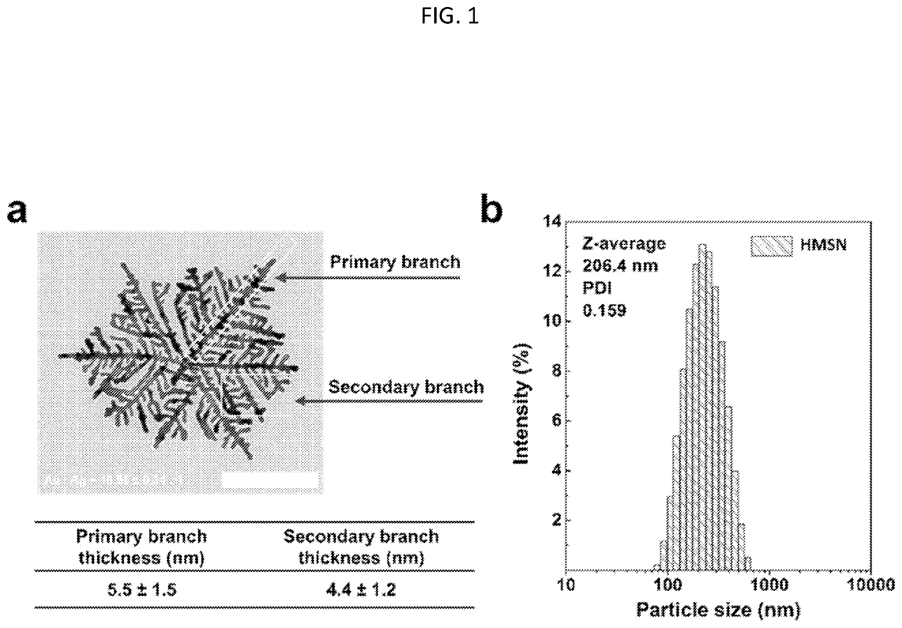

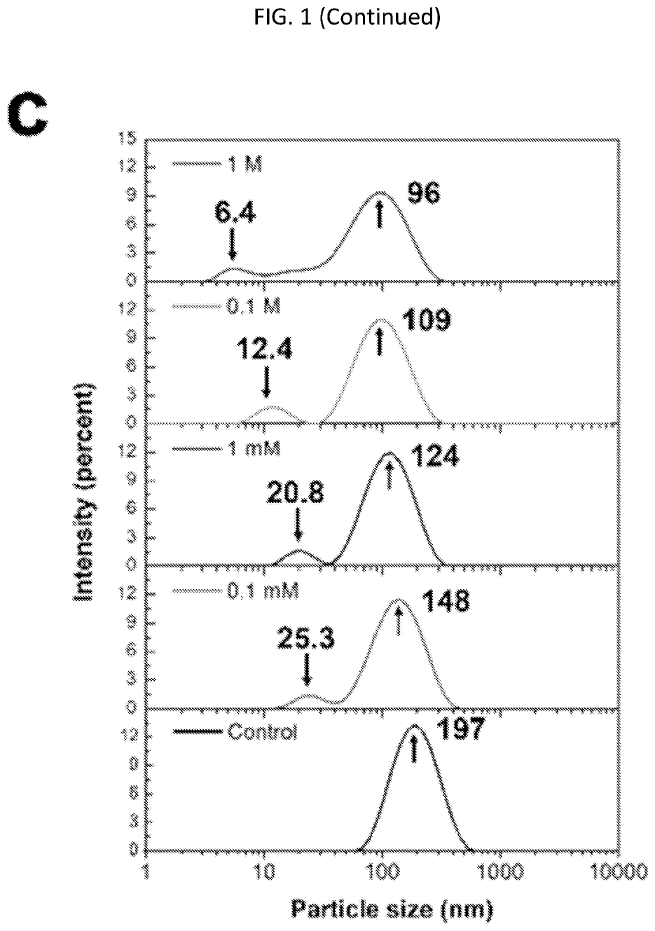

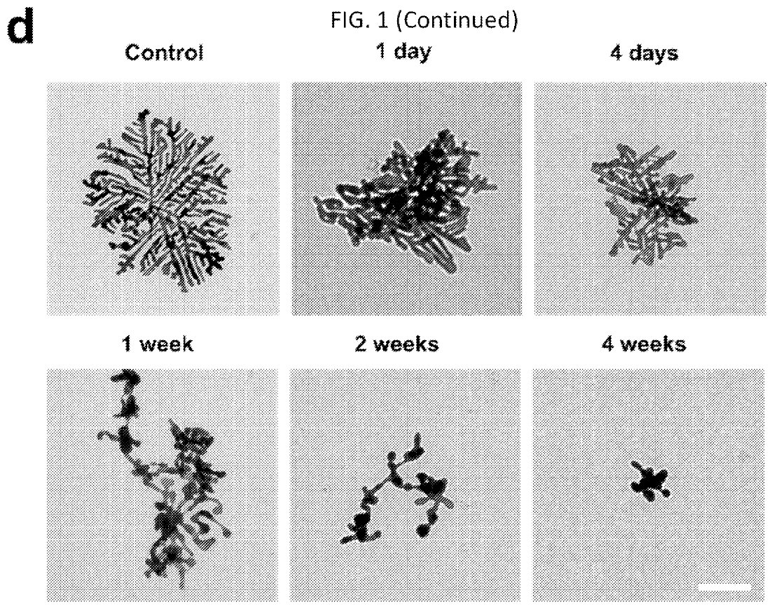

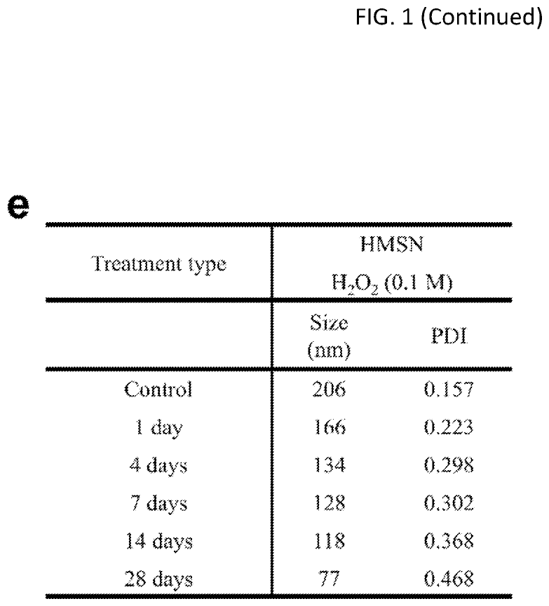

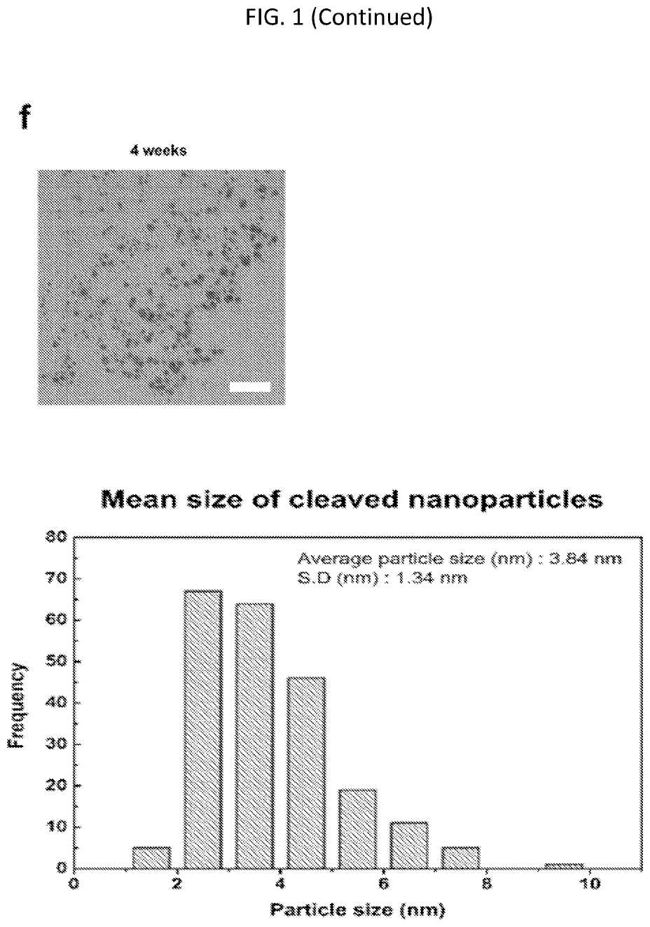

HMSN were synthesized with the mixture of 5:1 molar ratio of HAuCl₄.3H₂O and AgNO₃ in the 1.8 mM sodium cholate solution and 100 mM of 1-ascorbic acid for the potential ROS responsive cleavable Au nanocarriers (FIG. 1a). The preferential Au crystal growth in the structure of cholic acid formed the HMSN which was composed with multi-straight branches including ˜5.5 nm thickness major branches and ˜4.4 nm thickness secondary branches (FIG. 1a). Hydrodynamic size of HMSN was about 200 nm (FIG. 1b).

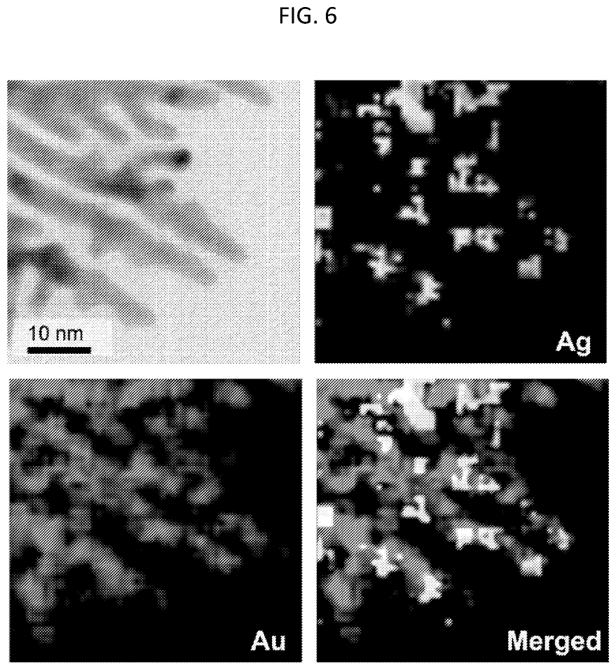

Unique alternating Au and Ag nanocrystal structure of HMSN allowed a significant structural deformation in the response to various H₂O₂ concentration. As shown in the DLS size distribution data of HMSN incubated in H₂O₂ solution for 7 days (FIG. 1c), the increase of H₂O₂ concentration induced the decrease of average size of HMSN from 197 nm to 96 nm. At the same time, the new peaks in the size distribution were appeared after the exposure to ROS of H₂O₂ solutions. Relatively low 0.1 mM H₂O₂ concentration generated 25.3 nm of the 2^{nd} peak. As the concentration of H₂O₂ increased to 1 M, 6.4 nm of 2^{nd} peak was observed. These results indicated ROS responsive cleavage of HMSN resulting in small nanoparticles appearance. Resulted ROS responsive structural change of HMSN was further confirmed with morphology change dependent on ROS exposure time. TEM data showed intact HMSN structure before the ROS exposure was significantly deformed within 1-day of ROS exposure with 0.1 M of H₂O₂. After 4 weeks of ROS exposure (H₂O₂ (0.1 M)), the outer part of HMSN was remarkably dissociated to be smaller sized nanostructures (FIGS. 1d and 1e). About ˜3.8 nm small nanoparticles, which was cleaved from HMSN, was primarily found in TEM images (FIG. 1f). Resulted ROS responsive degradability of HMSN was comparable with no significant size change of spherical Au nanoparticles (SGNP, 30 nm) during 4 weeks of ROS exposure (FIG. 6).

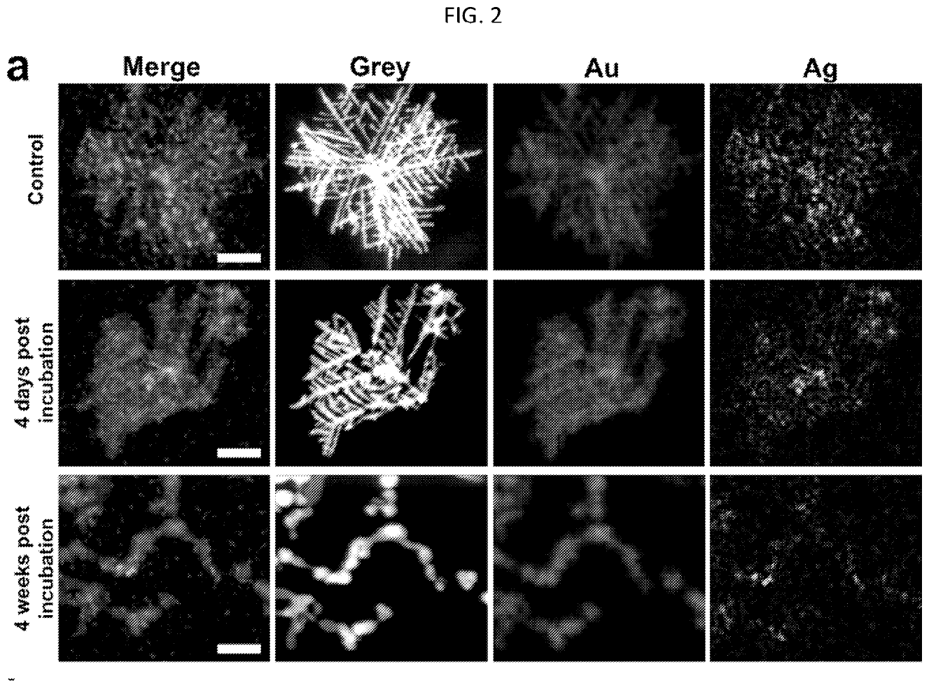

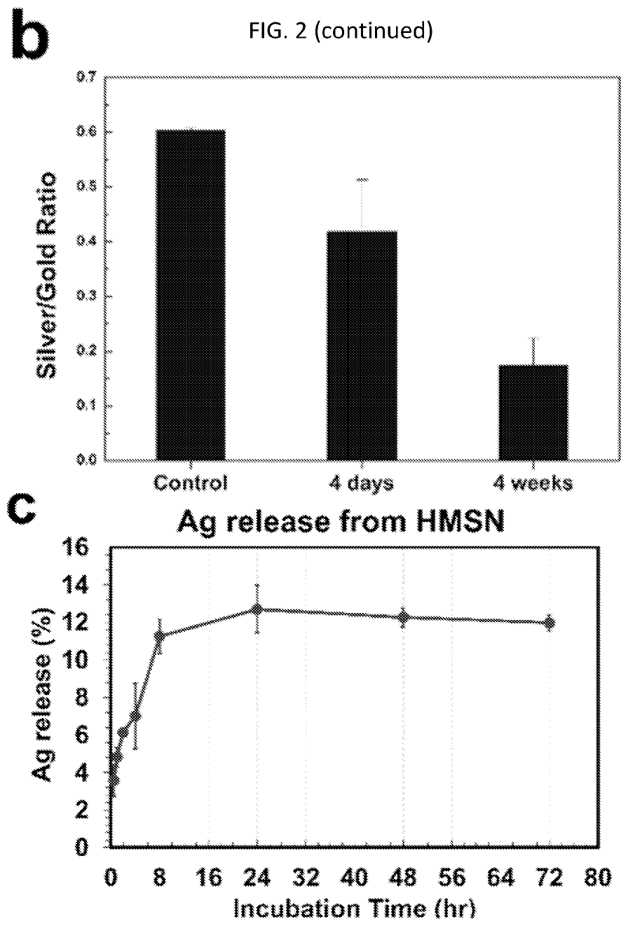

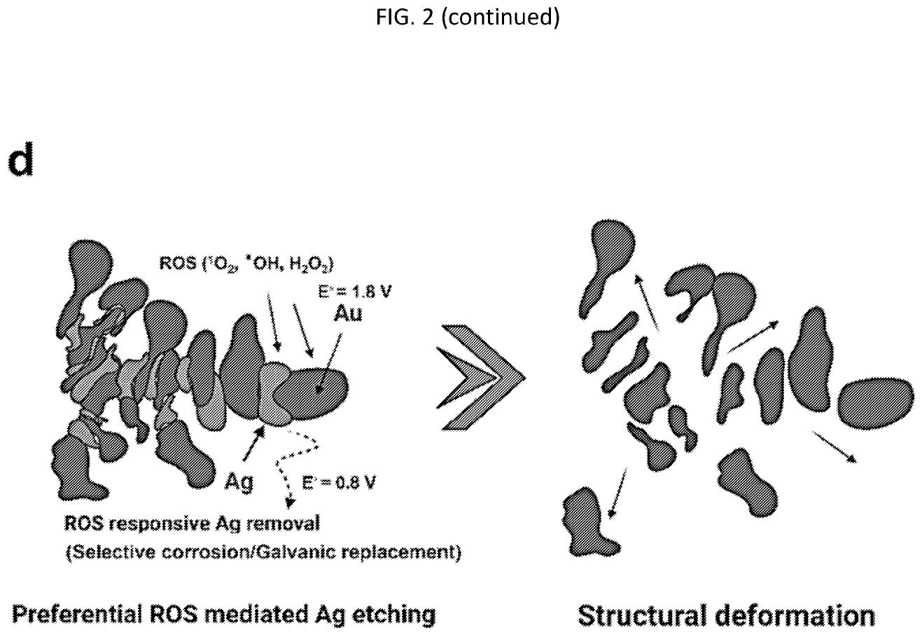

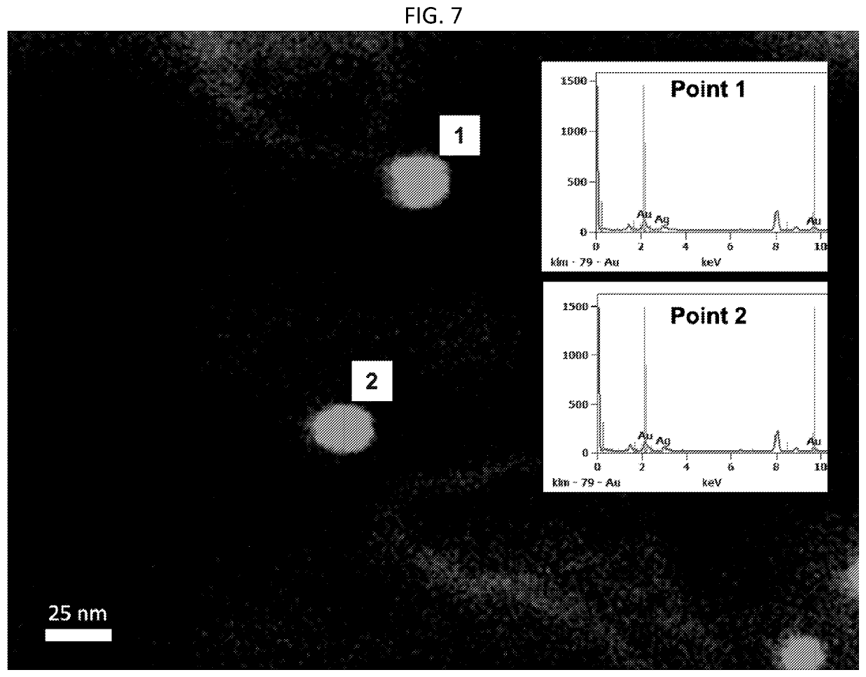

To further investigate the ROS responsive cleavage of HMSN, spatial distribution of Au and Ag elements in HMSN was measured with STEM. The STEM image of HMSN showed unique alternating Au and Ag nanostructures (FIG. 2a and FIG. 6). ICP-MS analysis indicated that the Au and Ag ratio of HMSN was 10.6:1. STEM images showed clear primary Au components (red) and secondary Ag components (green) of HMSN. However, the ROS exposure to the HMSN gradually leached out Ag components with structural dissociation (FIG. 2a). As shown in Ag/Au elemental intensity ratio, the Ag elemental intensity was significantly decreased at 4-day and 4-week post ROS treatment with H₂O₂ (FIG. 2b). This result was further confirmed with measuring the released silver component from HMSN by ICP-MS. Time dependent ROS responsive Ag release profile from HMSN showed 12% of Ag leaching of HMSN for 72 hours incubation with 0.1 M H₂O₂ solution (FIG. 2c). Additional STEM data showed that the remained HMSN structure after the ROS exposure was mainly composed by Au element with minor silver components (FIG. 7). These results indicate that HMSN branched structure composed by primary Au nanocrystals and Ag nano-linkers can be degradable with the selective ROS mediated etching of small Ag nanocrystal located between Au nanocrystals in HMSN (FIG. 2d). Relatively lower reduction potential of Ag component (E=0.80 V) in the Au nanocrystal (E=1.50 V) allowed the preferential ROS mediated Ag etching in the H₂O₂ solution. At the same time, galvanic replacement between Au and Ag can occur in the presence of H₂O₂ for the selective sacrifice of small Ag nano-linkers. Consequently, the structural deformation and degradation of HMSN was induced due to the selective dissolution of Ag nano-linkers in the presence of ROS.

### In Vitro Cellular Endogenous ROS Responsive Cleavage of HMSN

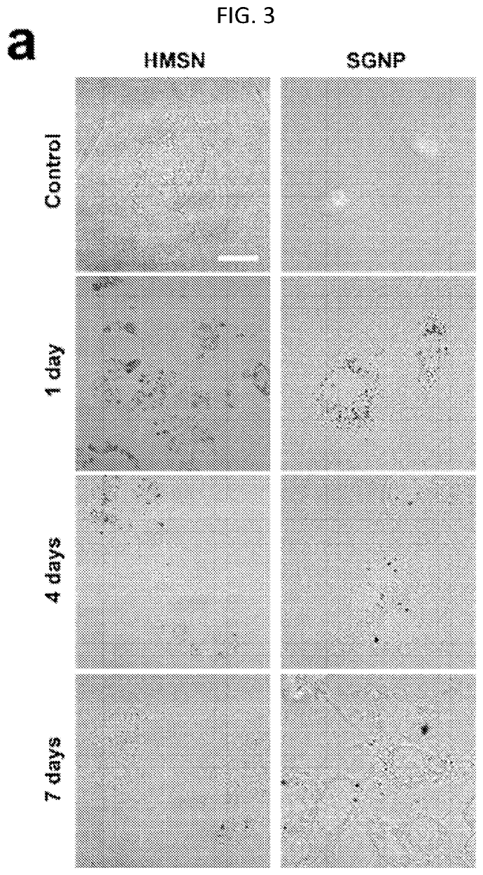

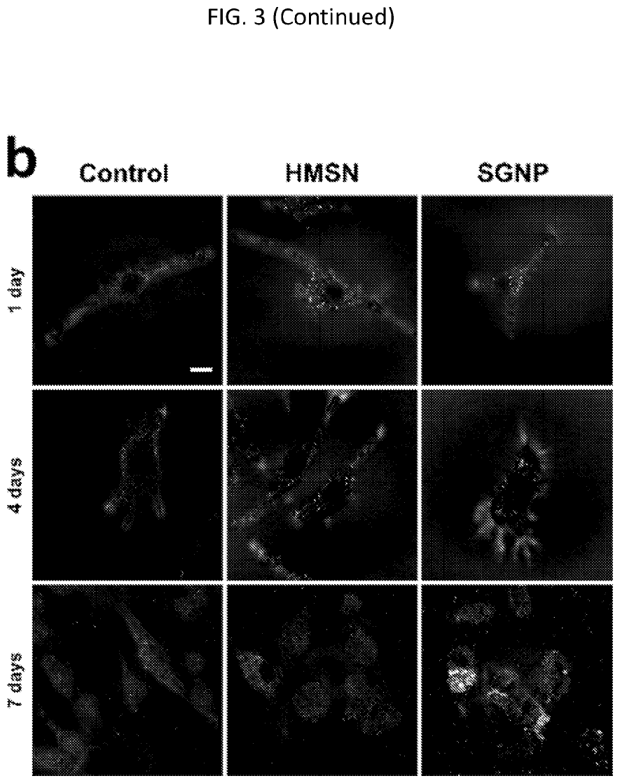

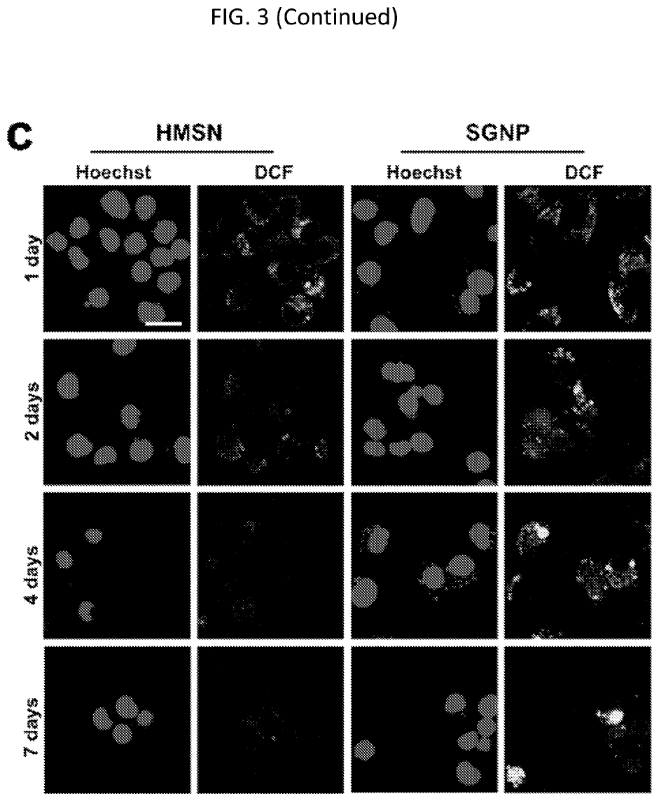

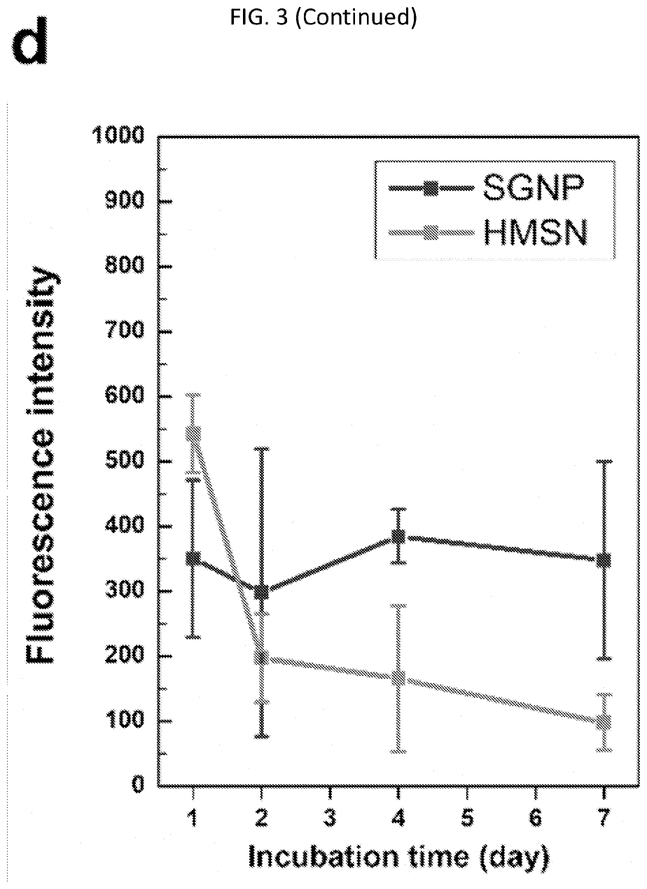

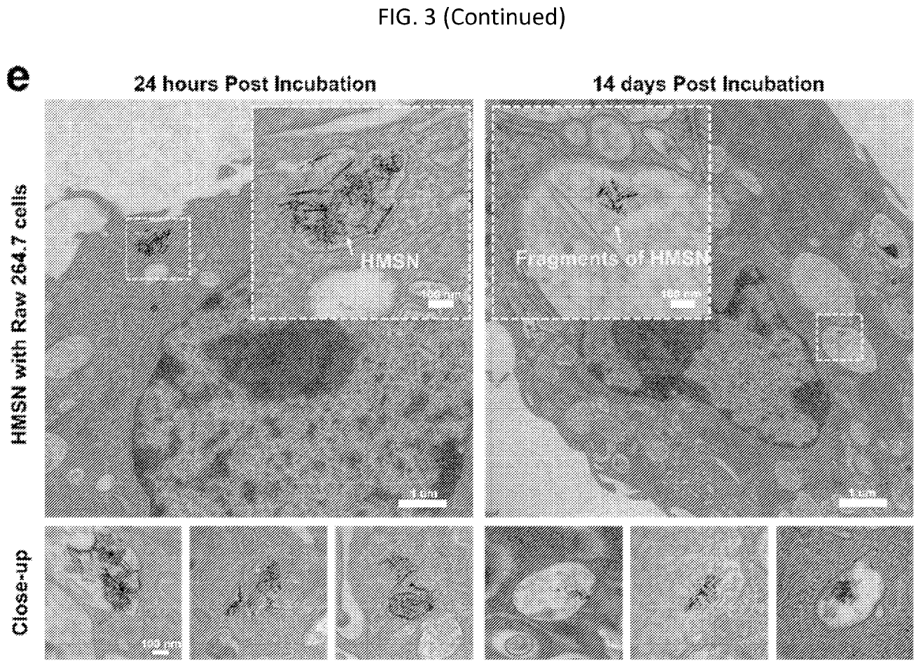

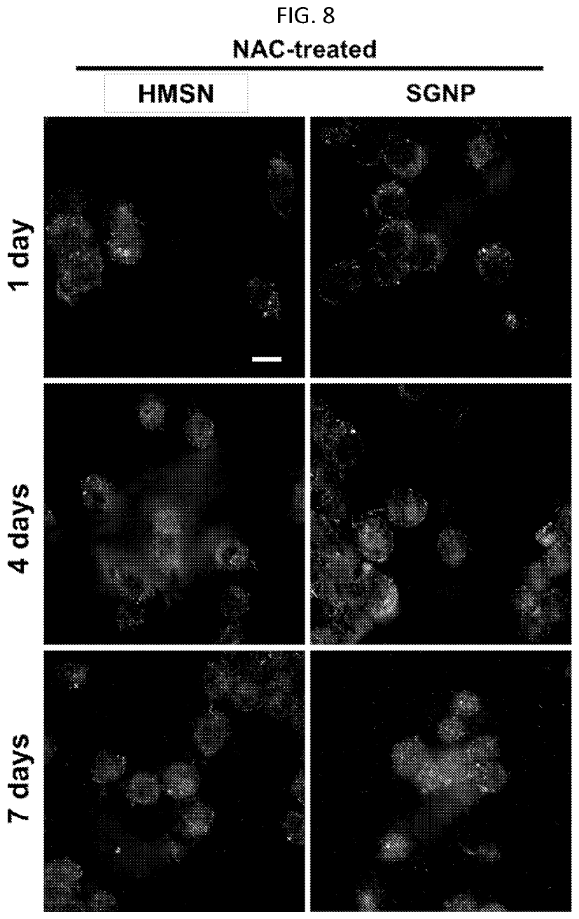

Demonstrated ROS responsive structural deformation with H₂O₂ solution was further studied for intracellular ROS responsiveness of HMSN using RAW 264.7 macrophage cells. HMSN were incubated with RAW 264.7 macrophage cells and their intracellular ROS responsive degradation was compared with a conventional spherical Au nanoparticle (SGNP) in confocal fluorescent microscope, confocal reflective microscope and cell TEM images. As shown in FIG. 3a, HMSN taken up by macrophage cells were clearly visible with black dots in confocal microscopic images at 1 day of post-incubation, as similar with SGNP treated cells. Those black dots indicating HMSN in cells were removed at 7-day post-treatment, while those SGNP treated cells still showed agglomerated nanoparticles. Dark field images of the cells showing gold nanoparticles also confirmed the significant signal reduction of HMSN at 7-day incubation (FIG. 3b). Intense and bright contrast of HMSN in cells at 1 day incubation was decreased and diluted at 7-day incubation period. However, the concentrated bright signal of SGNP was still visible in 7-day incubation. The significant exclusion of HMSN might be induced by intracellular ROS in the macrophages. Macrophages are phagocytes that can respond to nanoparticles by uptake and production of large quantity of ROS by respiratory burst to neutralize and digest nanoparticles in phagosomes or lysosomes. As shown in cellular ROS imaging of macrophages, each nanoparticles treatment significantly enhanced the ROS level in the cells. At 1-day post-treatment, strong ROS signals were observed in both HMSN similar with the SGNP treated cells. Then, the cells incubated with HMSN showed a significant ROS decrease from 2-day incubation. On 7-day incubation of HMSN, the cellular ROS level was dropped down to the similar ROS level of control group (FIGS. 3c and d). When a ROS inhibitor, N-acetyl cysteine, was treated in the cells, the clearance of HMSN was also inhibited. The reflectance signal from cells treated with HMSN were not decreased by time up to 7 days, indicating that the cells could not digest HMSN by the inhibited cellular ROS generation (FIG. 8). It is indicating that the intracellular endogenous ROS allowed the degradation of HMSN. Finally, the intracellular ROS cleavage of HMSN was further confirmed with TEM images of the cells incubated with HMSN. As shown in FIG. 3e, the branched structure of HMSN taken up by macrophages was significantly deformed to be dissociated into short branches in the hot ROS spots such as endosome or phagosome of cells. Longer incubation of HMSN with macrophage cells progressed the cleavage of HMSN, resulting the presence of smaller nano-fragments in intracellular vesicles corresponding to lysosome, or endosome, multivesicular bodies.

Although those significant removal of HMSN in the confocal microscope images can be involved exocytosis or physical exclusion, the significant size decrease and morphology change of HMSN in the intracellular vesicles might prove the endogenous ROS responsive cleavage and removal of HMSN. Taken together those cellular interaction data of HMSN, HMSN can be cleavable and degraded with the intracellular endogenous ROS, as demonstrated ROS responsive HMSN degradation in the H₂O₂ solution.

### Exogeneous ROS Responsive Degradability of HMSN

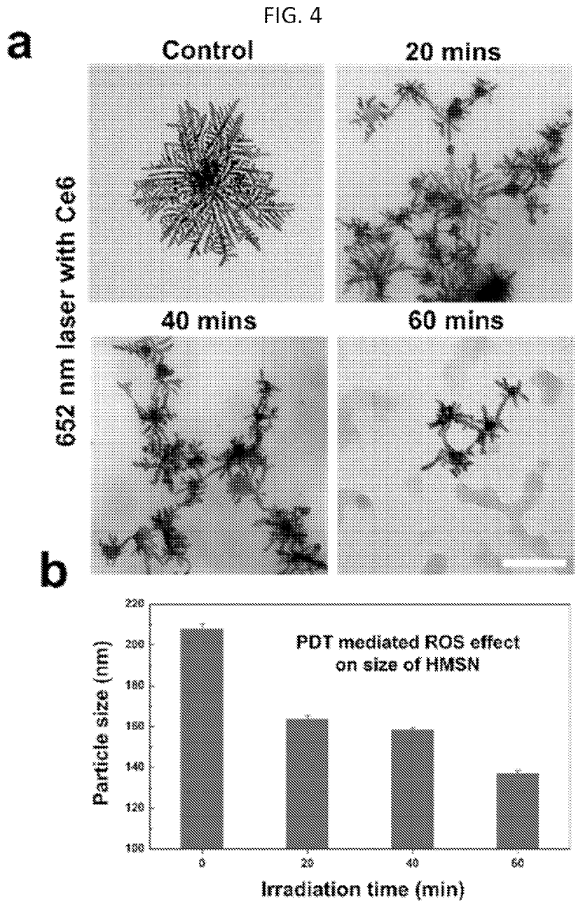

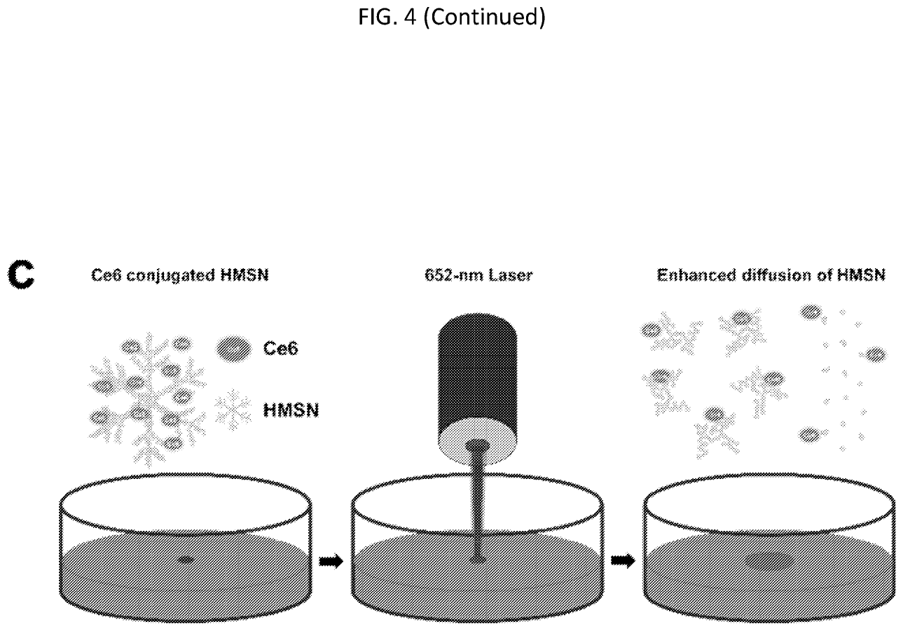

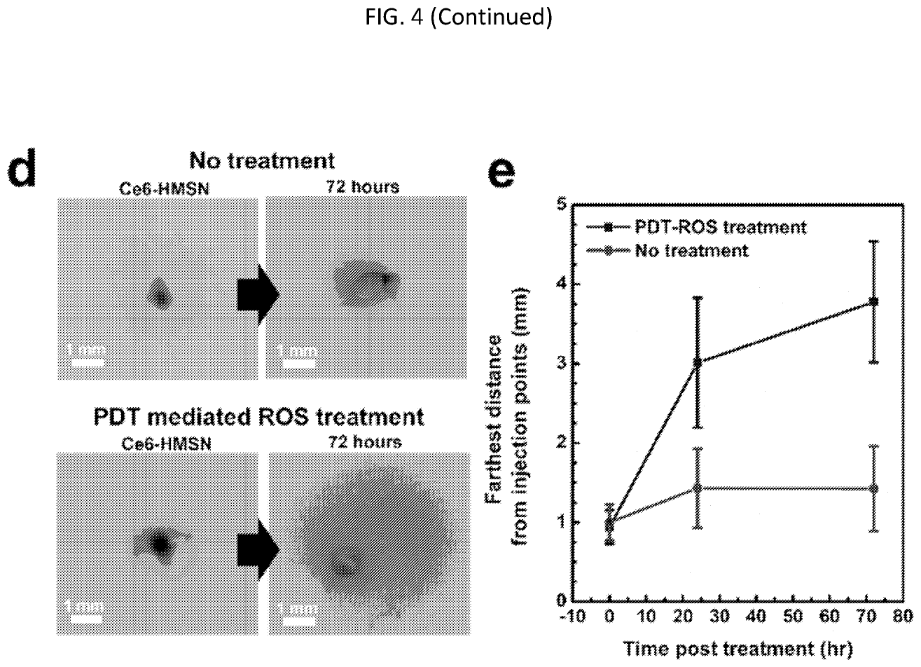

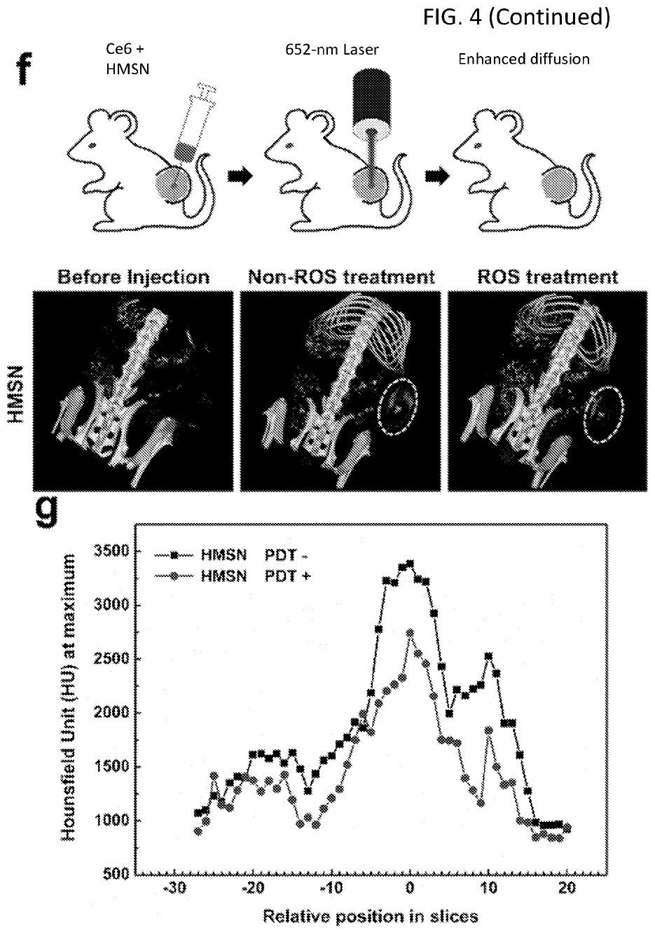

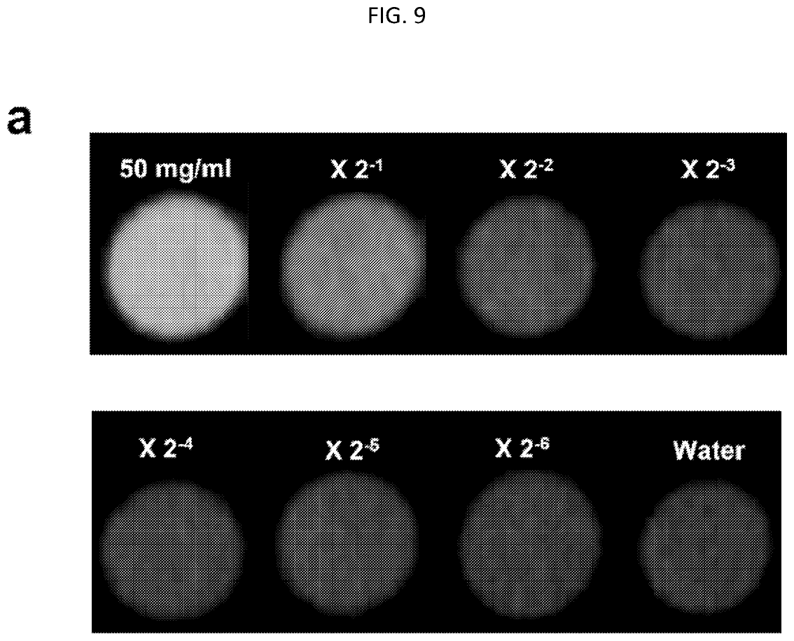

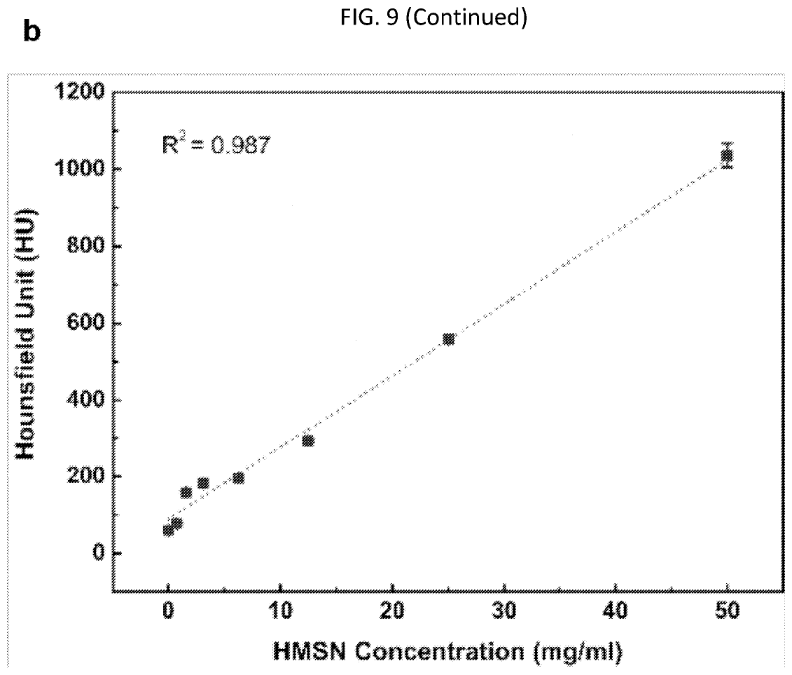

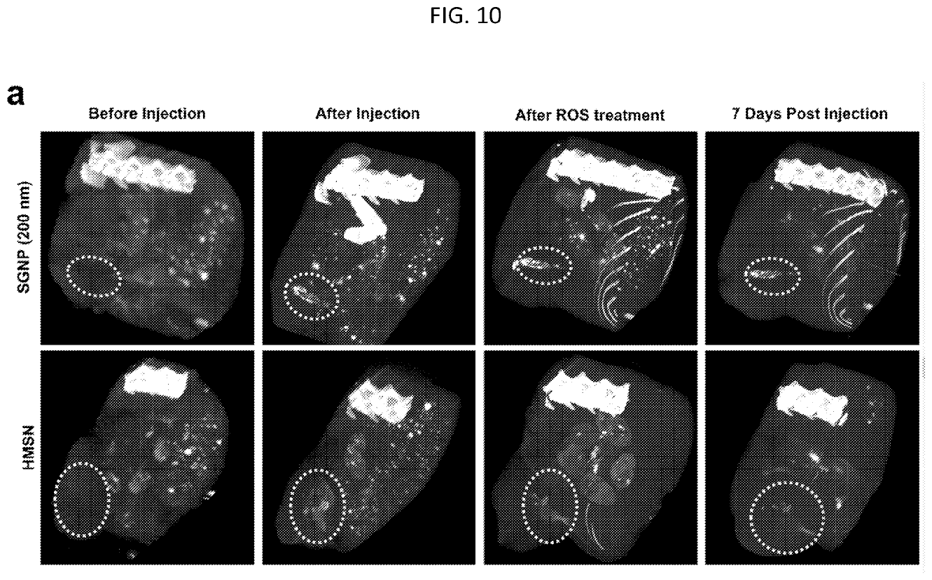

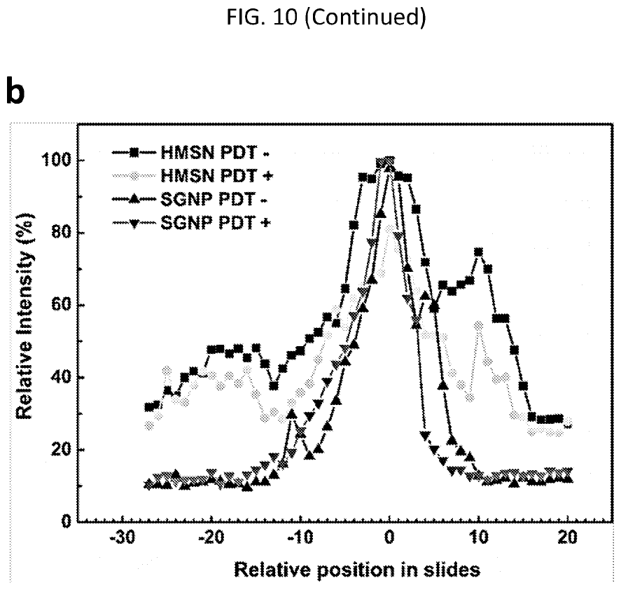

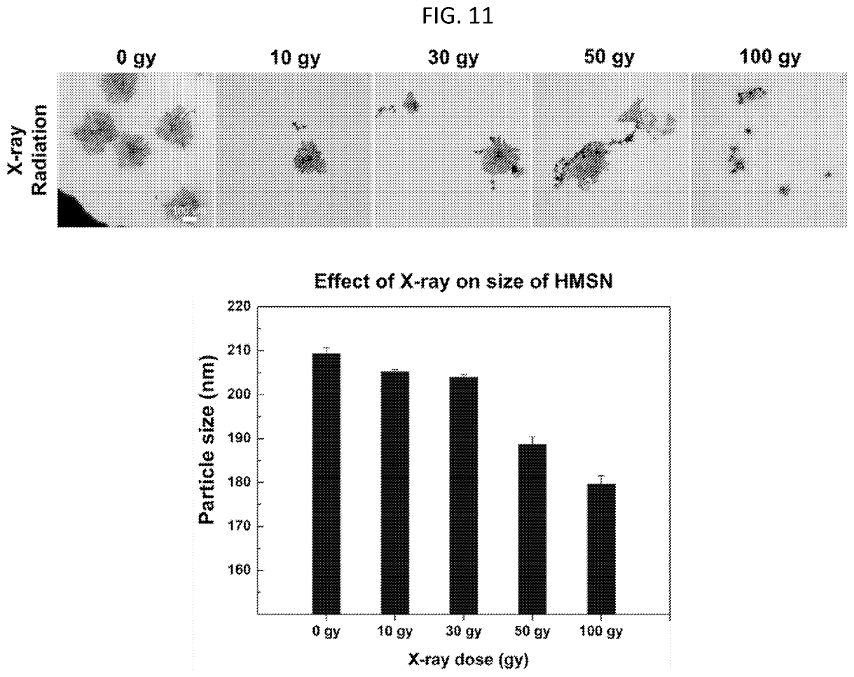

ROS responsive degradable HMSN can have great potential for various ROS mediated therapeutic applications such as PDT and radiation therapies. Next, therapeutic mediated exogenous ROS responsive degradability of HMSN was demonstrated with well-established PDT using Ce6 photosensitizers and X-ray radiation, respectively As shown in FIG. 4a, PDT mediated exogenous ROS significantly cleaved the branches of HMSN in the aqueous solution. The average size of HMSN (about 206 nm) was significantly decreased to 137 nm in the ROS exposure time dependent manner (FIG. 4b). The ROS mediated degradation of HMSN was further confirmed in a tissue phantom (1% agarose gel) mimicking a tissue environment. PDT mediated ROS was applied to HMSN implanted in tissue phantoms (FIG. 4c). Injected HMSN in the center of phantoms after 70 hours of PDT treatment were diffused 3-folds further distance from the injection center than non-ROS treated group. It is indicating the ROS responsive cleaved small branches of HMSN travels further with less hinderance in the gel network compared to non-ROS treated HMSN (FIGS. 4d and e). Subsequently, PDT mediated exogenous ROS responsive structural deformation and degradability of HMSN was tested in vivo with BALB/c mice bearing subcutaneously inoculated A20 tumor (FIG. 4f). HMSN with Ce6 was successfully injected into the center of tumor with innate CT contrast effect of HMSN (21 HU/mg/mL, FIG. 9) in CT scanning (FIG. 4f). Then, ROS were generated with the non-thermal laser (652 nm) for 30 mins. The distribution of injected HMSN in the tumor was measured with CT contrast changes at 1-day post ROS treatment (FIGS. 4f and 4g). PDT-ROS treated HMSN showed a significant reduction of CT contrast intensity (FIGS. 4f and 10). However, PDT-ROS treated SGNP showed no significant diffusion or reduction of CT contrast (FIG. 10). The demonstrated exogenous ROS responsive cleavage and degradability of HMSN will have a great potential for the combinational ROS mediated cancer therapeutic applications utilizing various established ROS cancer therapies such as PDT or radiation therapies (FIG. 11).

### In Vivo Biodistribution of Intravenously Injected ROS Responsive Degradable HMSN

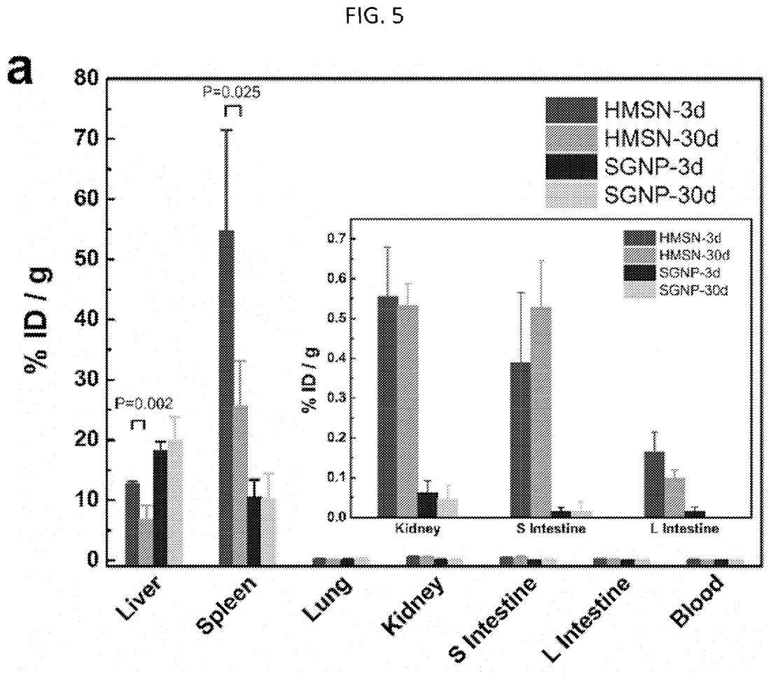

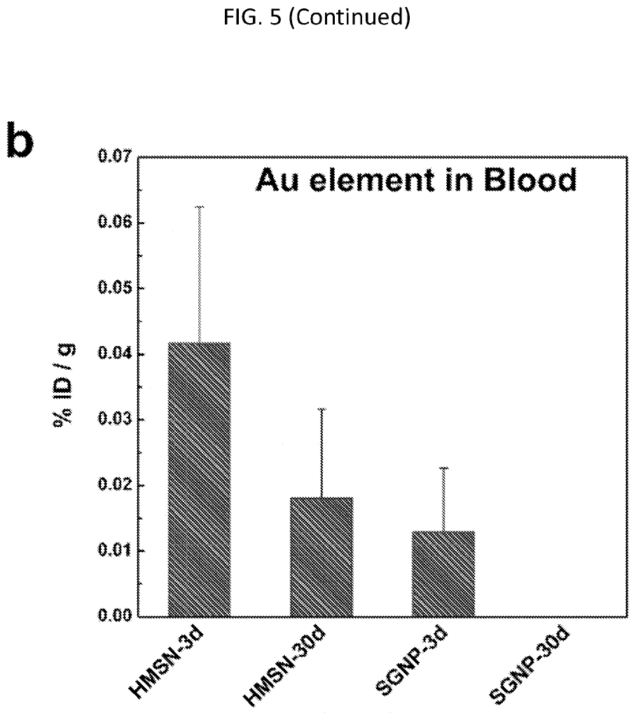

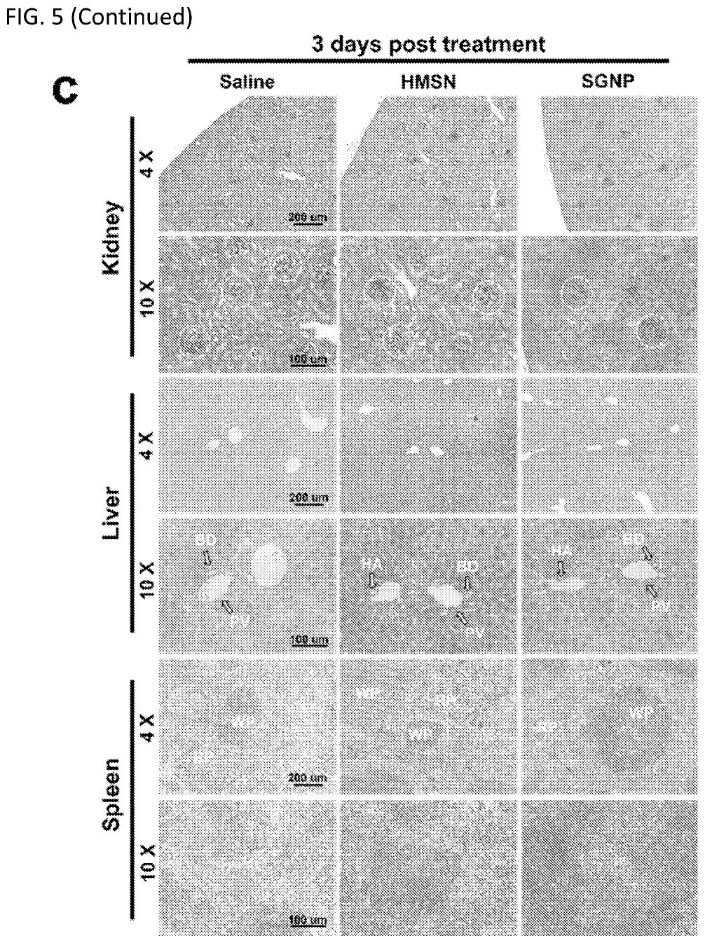

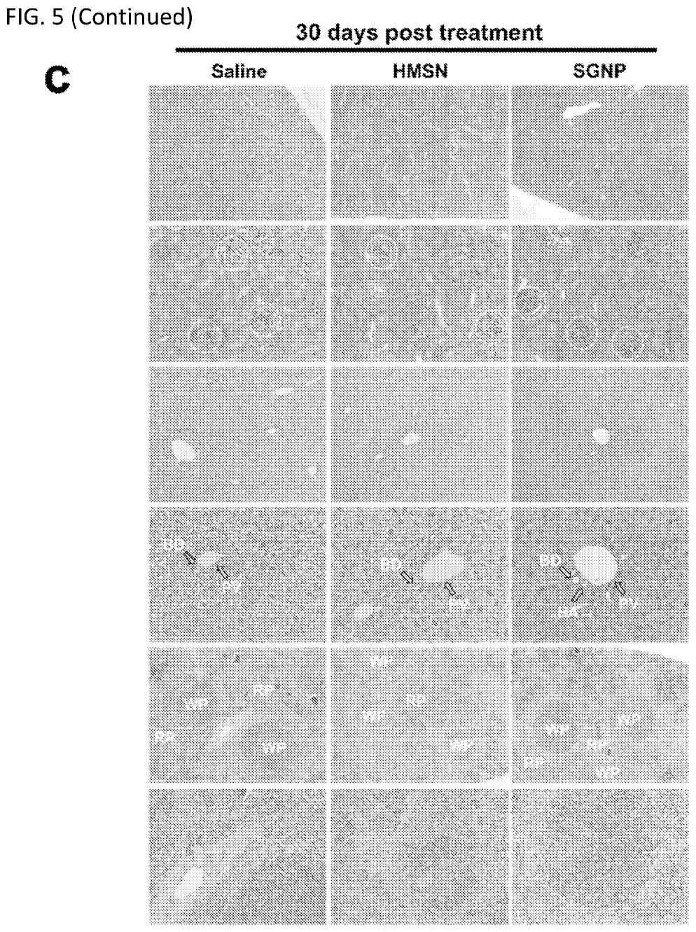

ROS responsive degradation of HMSN can be useful for the clearance of nanocarriers as well as local triggered delivery of therapeutics. As shown endogenous ROS responsive degradation of HMSN, IV injected HMSN can have enhanced clearance property compared to conventional non-degradable inorganic nanoparticles. As previously reported, the majority of the injected nanocarriers is cleared by cells such as macrophages in blood circulation and accumulated in the mononuclear phagocyte system (MPS) including the liver and spleen. The accumulated nanocarriers are going through ROS mediated digestion and the renal/biliary clearance. We hypothesized that our ROS responsive degradable HMSN can show faster clearance compared to non-degradable spherical gold nanoparticles (SGNP). Here, the biodistribution of IV injected HMSN was measured with Au elemental quantification in organs at 3-day and 30-day post injection and compared with the biodistribution of SGNP in C57BL/6 mice (FIG. 5). The quantification of Au element in each organ showed that IV injected HMSN were primarily accumulated in the liver and spleen as well as SGNP. However, the relative accumulation of HMSN in kidney (Ki), small intestine (SI) and large intestine (LI) was significantly higher than the mice treated with SGNP. This result might indicate the IV injected HMSN was degraded in the liver into smaller particles and secreted by hepatobiliary pathway more effectively than non-ROS responsive SGNP. Also, degraded small nanoparticles from HMSN may diffuse out from the organ and travel back to circulation. It is supported by the accumulation of Au in the kidney and the constant presence of Au element detected in blood (0.016% ID/g at 3 days and 0.018% ID/g 30 days post injection) throughout a month while the Au element was not detected in case of the mice treated with SGNP at 30 days post injection (FIG. 5b). Furthermore, at 30 days post injection, the liver and spleen accumulation of HMSN was decreased from the initial detected accumulations at 3 days post injection. On the contrary, SGNP injected mice showed no change of the Au accumulation in the liver and spleen. Also, SGNP injected mice did not show significant Au accumulation in their excretory organs (FIG. 5a). There are numerous distributional studies of spherical gold nanoparticles, demonstrating the retention time of non-degradable gold nanoparticles in vivo may be extremely long, e.g., more than a year without major deformation of nanoparticles. However, our ROS responsive degradability of HMSN suggest faster clearance by the biodistribution data. Our biodistribution data indicate potential renal clearance of our ROS responsive degradable HMSN that can mitigate long-term toxicity of the materials.

### In Vivo Biodistribution of Intravenously Injected ROS Responsive Degradable HMSN

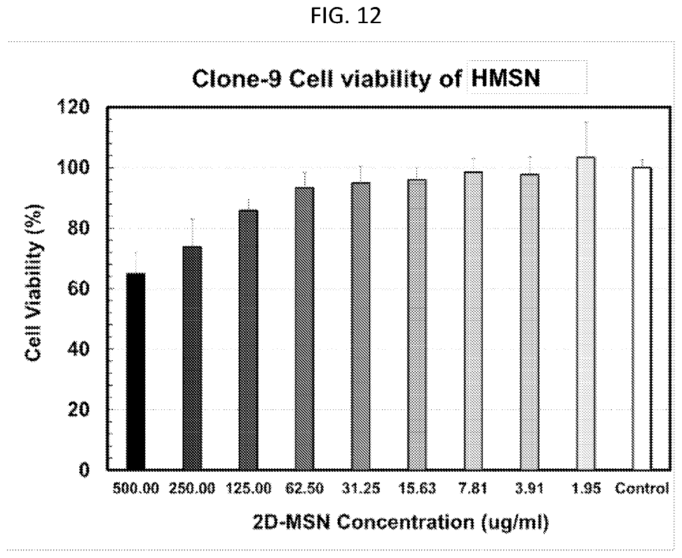

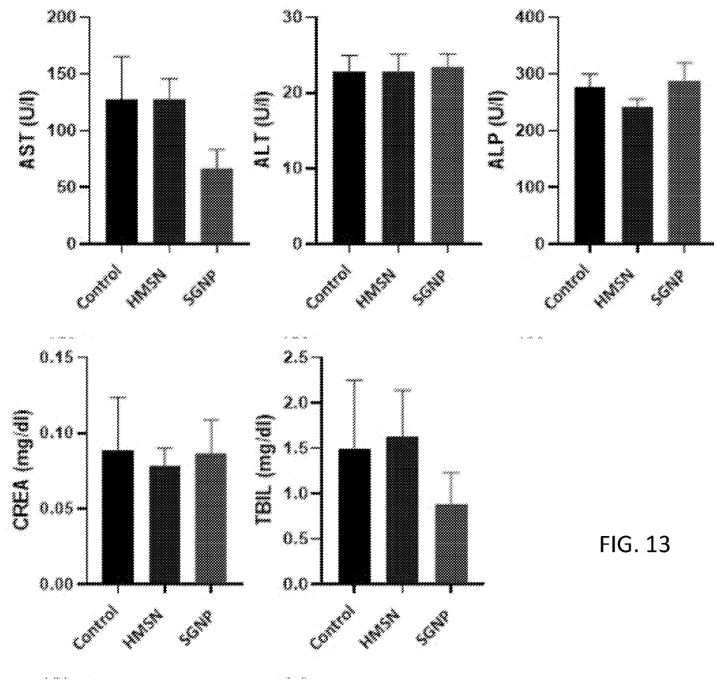

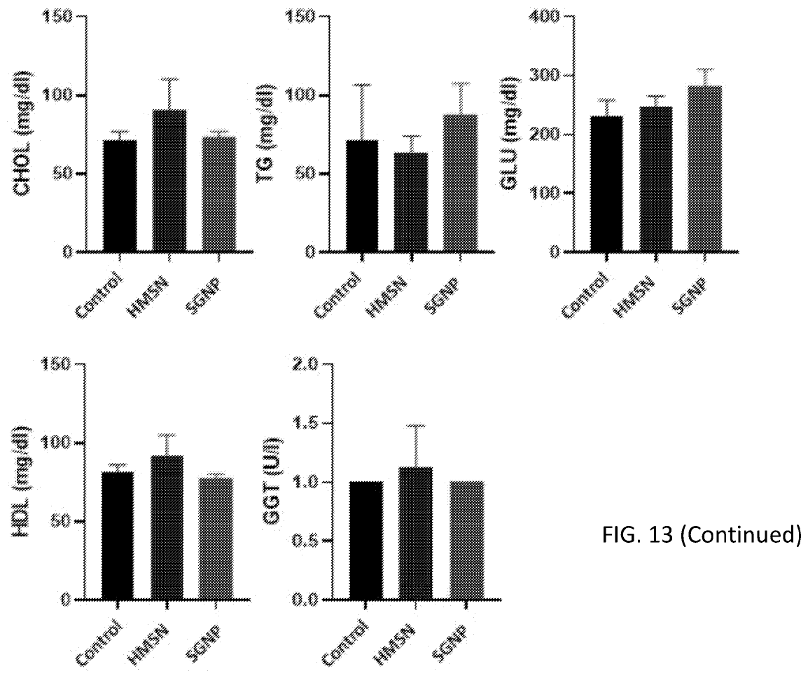

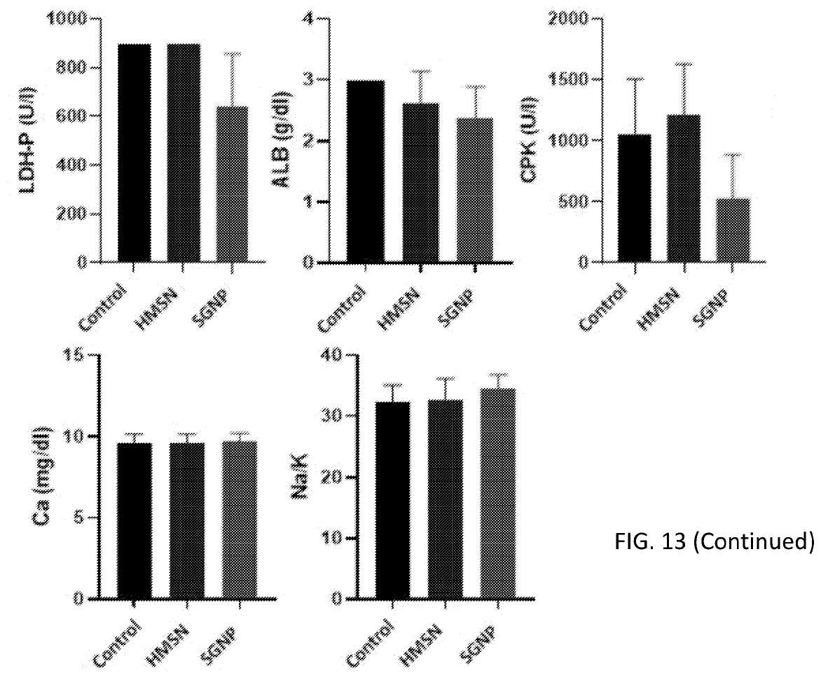

Although in vitro cytotoxicity assay of HMSN in Clone-9 hepatocytes showed no significant toxicity in a concentration range up to 250 μg/mL of HMSN (FIG. 12), in vivo safety of ROS responsive nanocarriers is an important component for the potential in vivo applications. During the biodistribution analysis, in vivo toxicity of HMSN was investigated together. During the treatment period by 30-day, the body weights of the mice treated with the nanoparticles had no appreciable change. No obvious histopathological abnormalities were found in these tissue sections at 7-day and 30-day, suggesting negligible adverse toxicity of HMSN (FIG. 5c). H&E histology data of organs from the mice treated with IV injection of 100 μg HMSN or SGNP at 3-day and 30-day post-injection showed no severe toxicity caused by injected nanoparticles in organs related to reticuloendothelial and excretory system. In kidney, focal and segmental glomerulosclerosis warning the renal toxicity was not found in kidney sections, and liver sections showed well-integrated structure of portal triad. Plus, there was no neutrophil infiltration and formation of foreign body giant cells in liver, and there was no significant geminal center maturation in spleen after treatment of HMSN compared to PBS treated control. The examination of the hematology values of mice treated with nanoparticles provided the information about potential liver- and kidney-function impairment. Compared to the control group, no significant changes in hematological parameters were found in HMSN injected animals as well as SGNP in our dosage (FIG. 13). There were no significant changes in AST and ALT, key markers of liver injury, on day 7-day post injection of HMSN and SGNP injection.

### Conclusion

The Examples demonstrate formation of HMSN structured with thin Au branches connected with Ag nano-linkers was synthesized. The ROS responsive cleavage of HMSN was demonstrated in various biological ROS conditions. The Examples show that Ag nano-linkers in widely exposed thin Au branches of HMSN was selectively removed in the response to a biological endogenous ROS and therapeutic exogeneous ROS condition. Due to well-known superior optical properties and catalytic activity of branched Au nanoparticles that can be utilized for sensing, imaging agents, and therapeutics and so on over spherical Au nanoparticles, HMSN allow for the ROS-responsive multifunctional nanocarriers. As given current challenges of non-degradable character and non-stimuli responsiveness of various potent inorganic nanoparticles for in vivo ROS-responsive nanocarrier applications, the introduction of ROS responsive cleavable HMSN allows for multifunctional inorganic nanoparticles for the ROS-responsive nanocarrier applications. Further, considering the impact of ROS biology, cellular oxidative stress, and ROS mediated therapeutics, ROS responsive cleavable HMSN allows for targeted drug delivery, localized combination immunotherapy, peroxidase-like nanozymes, sensors, and other advanced ROS related medical applications.

### Experimental Section

### Materials

Gold(III) chloride trihydrate, silver nitrate, l-ascorbic acid, sodium cholate hydrate, Milli-Q grade water, HS-PEG3500-COOH, HS-PEG3500-NH2, sodium citrate, 30% v/v hydrogen peroxide solution, Chlorine e6 (Ce6), N-(3-Dimethylaminopropyl)-N′-ethylcarbodiimide hydrochloride (EDC), N-hydroxysuccinimide (NHS), N-acetyl cysteine (NAC), and agarose were purchased from Sigma-Aldrich (Saint Louis, MO, USA). Chlorine e6 (Ce6) was purchased from Frontier Scientific (Logan, UT, USA).

### Synthesis of HMSN

10 mL of 1.8 mM sodium cholate solution was prepared in glass vial and stirred at 350 rpm. Then, 1 mL of 5 mM AuCl and 100 μL of 10 mM AgNO₃ solution was subsequently added into 20 second interval. 150 μL of 100 mM 1-ascorbic acid was added into reaction mixture and incubated for 20 second strictly at 350 rpm. Finally, the reaction mixture was left without agitation for 4 hours, and 100 μL of 100 mg/mL thiol-PEG-carboxylate was added to reaction mixture. The nanoparticle suspension was incubated for 12 hours. For purification, suspension of HMSN was centrifugated at 6500 rpm for 30 minutes, and supernatant solution was discarded. The purification step was repeated for 5 times. The synthesized HMSN was observed in transmission electron microscope (TEM) for demonstration of structure and was measured by dynamic light spectroscopy (DLS) for determination of size information. Quantification of gold and silver element in HMSN was measured with ICP-MS, and the elemental distribution in nanoparticles was acquired by EDS function with STEM.

### Demonstration of ROS Responsive Degradability of HMSN in H₂O₂

1 mL of HMSN suspension (100 μg/mL) was loaded in dialysis membrane (MW cut: 10 kD), and the membrane was incubated in H₂O₂ solution with various concentration (0, 0.1, 1, 100, and 1000 mM) for up to 4 weeks in 37° C. To maintain the initial concentration of H₂O₂ exposing to gold nanoparticles, the media outside of dialysis membrane was replaced with H₂O₂ solution freshly prepared from 30% v/v H₂O₂ stock solution. After the incubation, the suspension containing gold nanoparticles in dialysis membrane was collected and measured with TEM and DLS for morphological analysis and size determination, respectively. The elemental distribution of nanoparticles in the samples incubated in H₂O₂ solution was measured with STEM in predetermined time points (control, 4 days, 4 weeks). For quantification of silver ions released from HMSN during co-incubation with ROS source, 100 μL of 25 mg/ml. HMSN suspension was added into centrifugal tube containing 900 μL of 0.1 M H₂O₂. Hence, at predetermined time points, sample solutions were centrifugated using Amicon Ultra centrifugal filter (100 kD) (Millipore, MA, USA). The filtered solution was weighed and treated with trace metal grade nitric acid and hydrogen peroxide for ICP-MS analysis. The silver element in sample solution was measured using ICP-MS.

### In Vitro Intracellular Endogenous ROS Responsive Degradability of HMSN

RAW 264.7 macrophage cell line was used for the in vitro experiments. 4000 cells in 2 mL of media containing samples (non-treated, 10 μg/mL of HMSN, 10 μg/mL of SGNP) was seeded in culture dishes for confocal microscopy and incubated up to 7 days. For ROS generation in macrophages, after the incubation with nanoparticles in predetermined time (1, 2, 4, and 7 days), the media was discarded and replaced with the media containing 5 μM DCF2DA. After 40 minutes of incubation in incubation chamber, the cells were imaged by confocal microscope with excitation/emission at 485 nm/535 nm. For confocal reflectance microscopy, living macrophage cells in confocal dish were directly imaged by reflectance microscope (Nikon A1R) equipped with O₂ and CO₂ chamber. To determine the effect of ROS on degradation of HMSN, the group with treatment of N-acetyl cysteine (NAC) as a ROS inhibitor (4 mM) was tested. Macrophage cells were incubated with HMSN for 14 days, fixed with formaldehyde and glutaraldehyde solution, and centrifugated at 1500 rpm for 5 minutes. The supernatant was discarded and 200 μL of 3% agarose was added to the pellet and mixed gently. The block was dehydrated with ethanol (50, 75, 90, and 100% v/v) and sectioned with ultramicrotome for obtaining TEM sections. After the placement of sections on TEM grids, the cells and intracellular structures were measured by TEM.

### ROS Therapeutic Mediated Exogenous ROS Responsive Degradability of HMSN

For testing the exogenous ROS responsive degradability of HMSN, 500 μL of 10 μg/mL Ce6 (photosensitizer) was mixed with the suspension containing 500 μL of 100 μg/mL HMSN. Then, 652 nm wavelength laser was irradiated up to dosage of 450 J/cm² to sample in a dark room. Samples were collected after the treatment of various laser exposure condition at 150, 350, and 450 J/cm², and measured with TEM and DLS. X-ray irradiation was also tested for the exogenous ROS responsive degradability of HMSN, 100 μg/mL HMSN in Milli-Q grade water was irradiated with X-ray (up to 100 Gy), and samples were taken for TEM and DIS measurement to observe morphological changes and hydrodynamic size change respectively after ROS treatments.

### Exogenous ROS Responsive Degradability of HMSN in Artificial Tissue Phantom

Ce6 conjugated HMSN was synthesized with EDC/NHS conjugation method. Briefly, 2.4 mg of HMSN was resuspended with Amine-PEG-thiol (MW: 3500). 0.2 mg of Ce6 dissolved in 5 mL of DMSO was treated with 5 mg of EDC and 3 mg of NHS. Then, the mixture was mixed with gold suspension and incubated in a dark room at room temperature for 8 hours. Ce6 conjugated HMSN was purified with dialysis and centrifugation for further use. 1% w/v agarose gel was prepared in 6 well plates, and 5 μL of Ce6 conjugated HMSN (2 mg/mL) was seeded in the center of gel. The gel was treated with 652 nm laser (450 J/cm²) and the diffusivity of nanoparticle in gel was imaged with digital camera (ILCE-7C, Sony, Japan). The images were processed with threshold function of ImageJ software, and the average distance of nanoparticle diffusion from the seeded point were measured.

### In Vivo Exogenous ROS Responsive Degradability of HMSN in Tumor

All animal experiments were approved by institutional IACUC. Female BALB/c mice were used to test the enhanced diffusivity of nanoparticles by treatment of ROS induction by a photosensitizer (Ce6) and irradiation of 652-nm laser in vivo. 1×10⁶ cells of A20 (B cell lymphoma) cell line were inoculated subcutaneously near thigh of mice. After the tumor size reached to diameter of 1 cm, the 10 mg/kg of Ce6 and 500 μg of HMSN or Spherical gold nanoparticle (200 nm) were injected by intratumoral route. Then, 150 J/cm² of laser dose (652 nm) was treated on tumor. Each step was imaged with MicroCT (35 kV, 450 ms) for evaluation of gold distribution in the tumors. The acquired DICOM scans were processed with Radiant Viewer (Poland) to reconstruct 3-dimensional body images. The distribution of CT contrast among sections were analyzed by following methods. Firstly, maximum contrast effect (HU) of transverse CT sections were measured by elliptical method with the area of 0.03 cm². After all maximum values in selected area in each slice were obtained, the slice that showed highest maximum intensity by gold nanoparticles in tumor was positioned at zero, and the slices directed to cranial position were counted negatively (−), and the slices directed to caudal position were counted positively (+).

### Biodistribution and Histological Analysis, Hematological Response of IV Injected ROS Responsive Degradable HMSN

100 μg of nanoparticle suspensions (HMSN or SGNP) was intravenously injected via tail vein of C57BL/6 mice. The mice were sacrificed at predetermined time points, and organs were collected for biodistribution assay. The collected organs were digested with nitric acid and hydrogen peroxide at 70° C. for 12 hours. The gold in digested organ solution was dissolved with hydrochloric acid. The mass of elemental gold in organs were measured using ICP-MS. Also, cell viability was measured using CCK-8 assay kit (Dojindo) to evaluate possible toxicity of HMSN. To evaluate the tissue viability and response to nanoparticles, Hematoxylin-Eosin staining was performed to the tissue 30 days after I.V administration of 100 μg of gold nanoparticles (HMSN or SGNP). To determine possible toxicity, the serum was isolated from the blood of mice treated with PBS, 50 μg of SGNP, and 50 μg of HMSN via I.V injection.

### REFERENCES

[1] R. A. Cairns, I. S. Harris, T. W. Mak, Nature Reviews Cancer 2011, 11, 85.
[2] C. Gorrini, I. S. Harris, T. W. Mak, Nature reviews Drug discovery 2013, 12, 931.
[3] V. Nogueira, Y. Park, C.-C. Chen, P.-Z. Xu, M.-L. Chen, I. Tonic, T. Unterman, N. Hay, Cancer cell 2008, 14, 458.
[4] D. E. Dolmans, D. Fukumura, R. K. Jain, Nature reviews cancer 2003, 3, 380.
[5] A. K. Holley, L. Miao, D. K. St. Clair, W. H. St. Clair, Antioxidants & redox signaling 2014, 20, 1567.
[6] a) X. Xu, P. E. Saw, W. Tao, Y. Li, X. Ji, S. Bhasin, Y. Liu, D. Ayyash, J. Rasmussen, M. Huo, Advanced Materials 2017, 29, 1700141; b) H. Jin, T. Zhu, X. Huang, M. Sun, H. Li, X. Zhu, M. Liu, Y. Xie, W. Huang, D. Yan, Biomaterials 2019, 211, 68.
[7] a) A. Napoli, M. Valentini, N. Tirelli, M. Müller, J. A. Hubbell, Nature materials 2004, 3, 183; b) N. Ma, Y. Li, H. Xu, Z. Wang, X. J. J. o. t. A. C. S. Zhang, 2010, 132, 442; c) M. S. Shim, Y. Xia, Angewandte Chemie 2013, 125, 7064.
[8] a) S. Cho, W. Park, H. Kim, J. R. Jokisaari, E. W. Roth, S. Lee, R. F. Klie, B. Lee, D.-H. Kim, ACS applied nano materials 2018, 1, 4602; b) S. Cho, B. Lee, W. Park, X. Huang, D.-H. Kim, ACS applied materials & interfaces 2018, 10, 27570; c) D.-H. Kim, A. C. Larson, Biomaterials 2015, 56, 154.
[9] B. Choi, H. Choi, B. Yu, D.-H. Kim, ACS nano 2020, 14, 13115.
[10] H. Jang, D.-H. Min, ACS nano 2015, 9, 2696.
[11] a) H. Jang, Y.-K. Kim, H. Huh, D.-H. Min, ACS nano 2014, 8, 467; b) A. Abdal Dayem, M. K. Hossain, S. B. Lee, K. Kim, S. K. Saha, G.-M. Yang, H. Y. Choi, S.-G. Cho, International journal of molecular sciences 2017, 18, 120; c) A. Balfourier, N. Luciani, G. Wang, G. Lelong, O. Ersen, A. Khelfa, D. Alloyeau, F. Gazeau, F. Carn, Proceedings of the National Academy of Sciences 2020, 117, 103; d) A. M. Nowicka, U. Hasse, M. Hermes, F. Scholz, Angewandte Chemie 2010, 122, 1079.
[12] a) J. D. Pollock, D. A. Williams, M. A. Gifford, L. L. Li, X. Du, J. Fisherman, S. H. Orkin, C. M. Doerschuk, M. C. Dinauer, Nature genetics 1995, 9, 202; b) T. Decoursey, E. Ligeti, Cellular and Molecular Life Sciences CMLS 2005, 62, 2173.
[13] a) G. Sahay, D. Y. Alakhova, A. V. Kabanov, Journal of controlled release 2010, 145, 182; b) J. D. Lambeth, A. S. Neish, Annual Review of Pathology: Mechanisms of Disease 2014, 9, 119; c) A. Manke, L. Wang, Y. Rojanasakul, BioMed research international 2013, 2013.
[14] K. Tomankova, J. Horakova, M. Harvanova, L. Malina, J. Soukupova, S. Hradilova, K. Kejlova, J. Malohlava, L. Licman, M. Dvorakova, Food and Chemical Toxicology 2015, 82, 106.
[15] E. A. Sykes, Q. Dai, C. D. Sarsons, J. Chen, J. V. Rocheleau, D. M. Hwang, G. Zheng, D. T. Cramb, K. D. Rinker, W. C. Chan, Proceedings of the National Academy of Sciences 2016, 113, E1142.
[16] a) W. Park, S.-J. Park, S. Cho, H. Shin, Y.-S. Jung, B. Lee, K. Na, D.-H. Kim, Journal of the American Chemical Society 2016, 138, 10734; b) W. Park, S. Cho, J. Han, H. Shin, K. Na, B. Lee, D.-H. Kim, Biomaterials Science 2018, 6, 79.
[17] K. M. Tsoi, S. A. MacParland, X.-Z. Ma, V. N. Spetzler, J. Echeverri, B. Ouyang, S. M. Fadel, E. A. Sykes, N. Goldaracena, J. M. Kaths, Nature materials 2016, 15, 1212.
[18] W. Poon, Y.-N. Zhang, B. Ouyang, B. R. Kingston, J. L. Wu, S. Wilhelm, W. C. Chan, Acs Nano 2019, 13, 5785.
[19] a) E. Sadauskas, G. Danscher, M. Stoltenberg, U. Vogel, A. Larsen, H. Wallin, Nanomedicine: Nanotechnology, Biology and Medicine 2009, 5, 162; b) J. Kolosnjaj-Tabi, Y. Javed, L. Lartigue, J. Volatron, D. Elgrabli, I. Marangon, G. Pugliese, B. Caron, A. Figuerola, N. Luciani, ACS nano 2015, 9, 7925.

1. A hierarchical metallic supra-nanostructure (HMSN) comprising branches with alternating nanocrystals and nano-linkers, wherein the nanocrystals comprise a first metal and the nano-linkers comprise a second metal having a lower reduction potential than the nanocrystals.

2. The hierarchical metallic supra-nanostructure of claim 1, wherein the nano-linkers are selectively dissolved in the presence of a reactive oxygen species (ROS).

3. The hierarchical metallic supra-nanostructure of claim 1, wherein the first metal is gold and/or the second metal is silver.

4. The hierarchical metallic supra-nanostructure of claim 3, wherein the first metal is gold and the second metal is silver.

5. The hierarchical metallic supra-nanostructure of claim 1, wherein the hierarchical metallic supra-nanostructure comprises primary branches and secondary branches extending from the primary branches.

6. The hierarchical metallic supra-nanostructure of claim 5, wherein the primary branches have a greater thickness than the secondary branches.

7. The hierarchical metallic supra-nanostructure of claim 1, wherein the hierarchical metallic supra-nanostructure has a particle size from 20 nm to 1000 nm.

8. The hierarchical metallic supra-nanostructure of claim 1, wherein the hierarchical metallic supra-nanostructure has an average particle size from about 20 nm to 800 nm.

9. The hierarchical metallic supra-nanostructure of claim 1, wherein the nanocrystals have a particle size from 1 nm to 10 nm.

10. The hierarchical metallic supra-nanostructure of claim 1, wherein the hierarchical metallic supra-nanostructure is two-dimensional.

11. The hierarchical metallic supra-nanostructure of claim 1 further comprising an agent conjugated with the hierarchical metallic supra-nanostructure.

12. The hierarchical metallic supra-nanostructure of claim 11, wherein the agent is a photosensitizer.

13. A method for preparing the hierarchical metallic supra-nanostructure of claim 1 comprising preparing a reaction mixture by adding a nanocrystal precursor and nano-linker precursor to a metal cholate solution; adding a reducing agent to the reaction mixture; and preparing a nanoparticle suspension from the reduced reaction mixture.

14. The method of claim 13, wherein the nanocrystal precursor is AuCl, the nano-linker precursor is AgNO₃, the metal cholate is sodium cholate, the reducing agent is ascorbic acid, or any combination thereof.

15. The method of claim 13, wherein the nanoparticle suspension is prepared by adding a thiol-PEG-carboxylate to the reduced reaction mixture.

16. A method for treating a subject in need of a reactive oxygen species-mediated therapy, the method comprising administering the hierarchical metallic supra-nanostructure according to claim 1 and the reactive oxygen species-mediated therapy to the subject.

17. The method of claim 16, wherein administering the reactive oxygen species-mediated therapy comprises irradiating the subject.

18. The method of claim 16, wherein the subject is in need of a treatment for cancer.

19. The method of claim 18, wherein the method induces apoptosis, senescence, or cell cycle arrest in cancer cells.

20. (canceled)

21. A method for degrading the hierarchical metallic supra-nanostructure according to claim 1, the method comprising contacting the hierarchical metallic supra-nanostructure with a reactive oxygen species.

22-24. (canceled)
